(19) United States
(12) Patent Application Publication
(10) Pub. No.: US 2015/0139922 A1
(43) Pub. Date: May 21, 2015

(54) EXTRACTS OF DARLINGTONIA CALIFORNICA

(71) Applicant: Merck Patent GmbH, Darmstadt (DE)

(72) Inventor: Corinna WIRTH, Darmstadt (DE)

(73) Assignee: Merck Patent GmbH, Darmstadt (DE)

(21) Appl. No.: 14/492,788

(22) Filed: Sep. 22, 2014

Related U.S. Application Data
(62) Division of application No. 14/118,329, filed on Nov. 18, 2013.

(51) Int. Cl. A61K 8/97; A61Q 19/04

(52) U.S. Cl. CPC A61K 8/97; A61Q 19/04
USPC 424/59; 424/774

(57) ABSTRACT

The present invention relates to an extract from plant parts of Darlingtonia californica, to a process for the preparation thereof, to the use of the extract in cosmetic or dermatological preparations and as self-tanning agent, and to preparations comprising an extract from plant parts of Darlingtonia californica and the preparation thereof.

The present invention relates to an extract from plant parts of Darlingtonia californica, to a process for the preparation thereof, to the use of the extract in cosmetic or dermatological preparations and as self-tanning agent, and to preparations comprising an extract from plant parts of Darlingtonia californica and the preparation thereof.

The trend away from refined paleness towards “healthy, sportily tanned skin” has been uninterrupted for years. In order to achieve a tanned complexion, people expose their skin to sunlight, since this causes pigmentation due to melanin formation. However, the UV radiation in sunlight also has a damaging effect on the skin. Besides acute damage (sunburn), long-term damage occurs on excessive irradiation with light from the UVB region (wavelength 280-320 nm), such as, for example, an increased risk of contracting skin cancer. Excessive exposure to UVB and UVA radiation (wavelength: 320-400 nm) generates highly reactive free-radical species, which multiply further even after termination of the irradiation, and wrinkling and skin ageing occur as a consequence thereof.

Tanning (pigmentation) of the skin offers natural protection against the adverse consequences of sunlight. The epidermis contains individual pigment-forming cells, the melanocytes, besides the basal cells in its lowest layer, the basal layer. UV light stimulates the production of melanin in these cells, which is transported into the kerantinocytes (horny cells), where it becomes visible as a brown skin colour. Melanin protects the cell nuclei against further irradiation and the adverse effects it causes on the cell DNA.

Depending on the chemical composition of the pigments formed biochemically, a distinction is made between brownish-black eumelanin and reddish-yellow pheomelanin. The skin hue observed is determined by the ratio of these two types of melanin.

This pigment formation starting from the amino acid tyrosine is initiated predominantly by UVB radiation and is known as “indirect pigmentation”. Its development runs over a number of days; the suntan obtained in this way lasts a few weeks. In the case of “direct pigmentation”, which commences with the solar irradiation, predominantly colourless melanin precursors are oxidised by UVA radiation to dark-coloured melanin. Since this oxidation is reversible, it results in skin tanning which only lasts briefly.

Artificial tanning of the skin can be produced externally with the aid of make-up and orally by taking carotenoids.

Much more popular, however, is artificial tanning of the skin which can be achieved by the application of so-called self-tanners.

These compounds have, as a chemical structural feature, keto or aldehyde groups in the vicinity of alcohol functions and predominantly belong to the class of substances of the sugars. Particularly frequently employed self-tanning substances are 1,3-dihydroxyacetone (DHA), which is used in an amount of 700 t/a, and erythrulose.

Classical self-tanners can be reacted with the proteins and amino acids of the horny layer of the skin in the sense of a Maillard reaction or via a Michael addition, where polymers which give the skin a brownish hue form via a reaction route which has not yet been clarified completely. This reaction is complete after about 4 to 6 hours. The tan achieved in this way cannot be washed off and is only removed with the normal skin desquamation.

However, these coloured products do not themselves have UV-absorbent properties, meaning that additional sun protection (clothing, hat, UV filter) is necessary on exposure to the sun. In contrast to “sun-tanned” skin, skin tanned in this way is not protected against sunburn.

There is therefore a demand for dermatologically tolerated skin-colouring substances which are suitable for use in cosmetic and/or dermatological preparations or medical products and which enhance the natural tanning of the skin by increasing melanin synthesis and at the same time enable better inherent skin protection or sun protection, in particular against UVB radiation.

The object on which the present invention is based therefore consisted in the provision of novel self-tanning agents having improved properties.

Surprisingly, it has now been found that extracts from plant parts of Darlingtonia californica are suitable as self-tanning agent.

For the purposes of the invention, the term self-tanning active compound is used synonymously with self-tanning substance, self-tanner or self-tanner substance.

Darlingtonia californica (cobra lily) is a carnivorous plant which, as the only species in the Darlingtonia genus, is counted in the family of the pitcher plants (Sarraceniaceae).

Extracts of these plants are not known in the prior art:

EP 0249165 A2 describes the use of digestive juices of carnivorous plants such as Darlingtonia in medicaments for combating malignant and chronic diseases which directly or indirectly cause a change in the cell structures of the body cells.

US 2007/0122492 A1 discloses a method of identifying plant extracts which are capable of inhibiting various extracellular proteases, such as matrix metalloproteases (MMPs) or human leucocyte elastase (HLE). The Sarraceniaceae family is listed as possible plants to be investigated.

U.S. Pat. No. 6,350,594 B1 describes the isolation of plant rubbers from plant cells in suspension culture and the use thereof in cosmetics. A list of possible plants includes the Sarraceniaceae family.

The present invention therefore relates firstly to an extract from plant parts of Darlingtonia californica.

In principle, all above-ground parts of the plant can be utilised for the extract. Plant parts preferably employed are the leaves. The leaves of Darlingtonia californica are tubular (“tubular leaves”) and serve as traps for trapping insects.

The plant extract from Darlingtonia californica can be prepared by methods known to the person skilled in the art. A plant extract which is particularly highly suitable for the use according to the invention is obtainable by a) extraction of the plant parts of Darlingtonia californica with the aid of a solvent and b) removal of the solvent.

Accordingly, the invention also relates to a process for the preparation of an extract from plant parts of Darlingtonia californica, comprising steps a) extraction of the plant parts of Darlingtonia californica with the aid of a solvent and b) removal of the solvent.

The plant parts can be employed directly for the extraction in accordance with step a). Typically, however, the plant parts are firstly dried and comminuted.

The drying can be carried out, for example, in air or preferably in a drying cabinet at elevated temperature, preferably between 30 and 50° C., particularly preferably at about 40° C. In addition, the drying can also be carried out under reduced pressure.

The comminution can be carried out by means of a standard method, for example with the aid of cutting tools, such as knives or scissors, or with the aid of a mixer.

The plant parts are subsequently extracted. The extraction is carried out with the aid of methods which are known to the person skilled in the art. For this purpose, a solvent is added to the plant parts.

Preference is given to the use of a solvent selected from water, organic solvents or mixtures thereof. Examples of organic solvents are methanol, ethanol, 1-propanol, 2-propanol, acetonitrile, acetone or ethyl acetate. Solvent mixtures comprising water and organic solvents preferably comprise 10-90% by vol. of water, particularly preferably 30-70% by vol. of water.

In a preferred embodiment of the present invention, the solvent is selected from water or alcohol. In a particularly preferred embodiment, an alcohol, in particular ethanol, is used as solvent.

The extraction is typically carried out at temperatures between 20 and 90° C., preferably between 50 and 90° C., particularly preferably at 70° C.

In step b), the solvent is removed. This is typically carried out by filtration.

The extract obtained in this way can optionally be purified further. For this purpose, methods familiar to the person skilled in the art can be employed, for example chromatographic methods, such as solid-phase adsorption with subsequent stepwise desorption by means of suitable solvents. The chromatography can also be carried out in countercurrent. Concentration and enrichment can also be carried out by means of supercritical gases or ionic liquids. In addition, it is possible to achieve enrichment of undesired constituents by liquid/liquid extraction or also to remove undesired constituents by changing the solvent with subsequent filtration.

The extract obtained can optionally subsequently be concentrated and/or dried in a step c).

This can be carried out using methods familiar to the person skilled in the art. The concentration is carried out, for example, using a rotary evaporator or a falling-film evaporator, and can also be carried out under reduced pressure. The drying of the extract can be carried out, for example, with the aid of spray drying or freeze drying. The extract is preferably dried completely to constant weight.

Extracts from plant parts of Darlingtonia californica have very good tanning properties. It is thought, without being tied to this theory, that the high tanning activity of the extracts according to the invention is attributable to a combination of various active components in the extracts. The tanning action of the extract is particularly good if it is prepared by the method described above and in particular by preferred embodiments thereof. The extracts according to the invention are eminently suitable for incorporation into cosmetic or dermatological preparations, in particular also owing to their neutral colour, which does not markedly colour the preparation. The preparation of the extract here is very environmentally friendly, since only small amounts of solvent have to be employed and no further chemicals are necessary.

The present invention therefore furthermore relates to the use of an extract from plant parts of Darlingtonia californica, as described above, in cosmetic or dermatological preparations.

The present invention likewise reates to the use of an extract from plant parts of Darlingtonia californica as described above as self-tanning agent.

In accordance with the invention, the use is preferably for increasing melanin synthesis, improving melanin transport and/or improving the distribution of melanin in suprabasal layers.

The extracts according to the invention increase melanin synthesis and improve melanin transport from the melanocytes to the keratinocytes. This has an effect on the colour of the skin and causes a tanning effect.

The use according to the invention preferably takes place non-therapeutically.

The present invention furthermore relates to a preparation comprising an extract from plant parts of Darlingtonia californica as described above.

The preparations here are usually preparations which can be applied topically, for example cosmetic or dermatological formulations or medical products. In this case, the preparations comprise a cosmetically or dermatologically suitable vehicle and, depending on the desired property profile, optionally further suitable ingredients. In the case of pharmaceutical preparations, the preparations in this case comprise a pharmaceutically tolerated vehicle and optionally further pharmaceutical active compounds.

In the sense of the present invention, the term formulation is also used synonymously alongside the term preparation.

“Can be applied topically” in the sense of the invention means that the preparation is used externally and locally, i.e. that the preparation must be suitable for, for example, application to the skin.

The preparations may include or comprise, essentially consist of or consist of the said requisite or optional constituents. All compounds or components which can be used in the preparations are either known and commercially available or can be synthesised by known processes.

The preparation is preferably a cosmetic or pharmaceutical preparation; the preparation is particularly preferably a cosmetic preparation.

The preparations according to the invention preferably comprise 0.01 to 99% by weight of the extract from Darlingtonia californica, based on the total weight of the preparation. An amount of 0.05 to 30% by weight is preferably employed, particularly preferably 0.1 to 10% by weight. The person skilled in the art is presented with absolutely no difficulties here in selecting the amounts correspondingly depending on the intended action of the preparation.

Preferred preparations according to the invention may comprise at least one further self-tanning substance as further ingredient. This can be either a self-tanner which reacts with the amino acids of the skin in the sense of a Maillard reaction or via a Michael addition, or a so-called melanogenesis promoter or propigmentation active compound which promotes the natural tanning of the skin.

Advantageous self-tanning substances which can be employed are, inter alia: 1,3-dihydroxyacetone, glycerolaldehyde, hydroxymethylglyoxal, ydialdehyde, erythrulose, 6-aldo-D-fructose, ninhydrin, 5-hydroxy-1,4-naphtoquinone (juglone) or 2-hydroxy-1,4-naphtoquinone (lawsone). Very particular preference is given to 1,3-dihydroxyacetone, erythrulose or a combination thereof.

Propigmentation substances can in principle be all active compounds known to the person skilled in the art. Examples thereof are glycyrrhetinic acid, melanocyte-stimulating hormone (alpha-MSH), peptide analogues, thymidine dinucleotides, L-tyrosine and esters thereof or bicyclic monoterpenediols (described in Brown et al., Photochemistry and Photobiology B: Biology 63 (2001) 148-161).

The at least one further self-tanning substance is preferably present in the preparation in an amount of 0.01 to 20% by weight, particularly preferably in an amount of 0.5 to 15% by weight and very particularly preferably in an amount of 1 to 8% by weight, based on the total amount of the preparation.

Preparations having self-tanner properties, in particular those which comprise dihydroxyacetone, tend towards malodours on application to the human skin, which are thought to be caused by degradation products of dihydroxyacetone itself or by products of side reactions and which are regarded as unpleasant by some users. It has been found that these malodours are prevented on use of formaldehyde scavengers and/or flavonoids. The preparation according to the invention may therefore preferably also comprise formaldehyde scavengers and optionally flavonoids for improving the odour.

The preparation according to the invention, which combines a further self-tanning substance and an extract from plant parts of Darlingtonia californica, has the following advantages over a self-tanning product without addition of the extract according to the invention:
extension of the tanning reaction owing to the indirect tanning reaction (UV-free tanning extension),
intensification of the tanning reaction,
prevention of uneven tanning due to inexpert application,
the tanning achieved comes close to natural tanning,
improvement in protection against UV radiation.

Besides the extracts according to the invention, the preparations according to the invention may additionally also comprise at least one UV filter.

Organic UV filters, so-called hydrophilic or lipophilic sun-protection filters, which are effective in the UVA region and/or UVB region and/or IR and/or VIS region (absorbers). These substances can be selected, in particular, from cinnamic acid derivatives, salicylic acid derivatives, camphor derivatives, triazine derivatives, β,β-diphenylacrylate derivatives, p-aminobenzoic acid derivatives and polymeric filters and silicone filters, which are described in the application WO-93/04665. Further examples of organic filters are indicated in the patent application EP-A 0 487 404. The said UV filters are usually named below in accordance with INCI nomenclature.

Particularly suitable for a combination are:

para-aminobenzoic acid and derivatives thereof: PABA, Ethyl PABA, Ethyl dihydroxypropyl PABA, Ethylhexyl dimethyl PABA, for example marketed by ISP under the name “Escalol 507”, Glyceryl PABA, PEG-25 PABA, for example marketed under the name “Uvinul P25” by BASF.

Salicylates: Homosalate marketed by Merck under the name “Eusolex HMS”; Ethylhexyl salicylate, for example marketed by Symrise under the name “Neo Heliopan OS”, Dipropylene glycol salicylate, for example marketed by Scher under the name “Dipsal”, TEA salicylate, for example marketed by Symrise under the name “Neo Heliopan TS”.

β,β-Diphenylacrylate derivatives: Octocrylene, for example marketed by Merck under the name “Eusolex® OCR”, “Uvinul N539” from BASF, Etocrylene, for example marketed by BASF under the name “Uvinul N35”.

Benzophenone derivatives: Benzophenone-1, for example marketed under the name “Uvinul 400”; Benzophenone-2, for example marketed under the name “Uvinul D50”; Benzophenone-3 or Oxybenzone, for example marketed under the name “Uvinul M40”; Benzophenone-4, for example marketed under the name “Uvinul MS40”; Benzophenone-9, for example marketed by BASF under the name “Uvinul DS-49”, Benzophenone-5, Benzophenone-6, for example marketed by Norquay under the name “Helisorb 11”, Benzophenone-8, for example marketed by American Cyanamid under the name “Spectra-Sorb UV-24”, Benzophenone-12 n-hexyl 2-(4-diethylamino-2-hydroxybenzoyl)benzoate or 2-hydroxy-4-methoxybenzophenone, marketed by Merck, Darmstadt, under the name Eusolex® 4360.

Benzylidenecamphor derivatives: 3-Benzylidenecamphor, for example marketed by Chimex under the name “Mexoryl SD”, 4-Methylbenzylidenecamphor, for example marketed by Merck under the name “Eusolex 6300”, benzylidenecamphorsulfonic acid, for example marketed by Chimex under the name “Mexoryl SL”, Camphor benzalkonium methosulfate, for example marketed by Chimex under the name “Mexoryl SO”, terephthalylidenedicamphorsulfonic acid, for example marketed by Chimex under the name “Mexoryl SX”, Polyacrylamidomethylbenzylidenecamphor marketed by Chimex under the name “Mexoryl SW”.

Phenylbenzimidazole derivatives: phenylbenzimidazolesulfonic acid, for example marketed by Merck under the name “Eusolex 232”, disodium phenyl dibenzimidazole tetrasulfonate, for example marketed by Symrise under the name “Neo Heliopan AP”.

Phenylbenzotriazole derivatives: Drometrizole trisiloxane, for example marketed by Rhodia Chimie under the name “Silatrizole”, Methylenebis(benzotriazolyl)tetramethylbutylphenol in solid form, for example marketed by Fairmount Chemical under the name “MIXXIM BB/100”, or in micronised form as an aqueous dispersion, for example marketed by BASF under the name “Tinosorb M”.

Triazine derivatives: Ethylhexyltriazone, for example marketed under the name “Uvinul T150” by BASF, Diethylhexylbutamidotriazone, for example marketed under the name “Uvasorb HEB” by Sigma 3V, 2,4,6-tris(diisobutyl 4′-aminobenzalmalonate)-s-triazine or 2,4,6-Tris(biphenyl)-1,3,5-triazine. marketed as Tinosorb A2B by BASF, 2,2′-[6-(4-methoxyphenyl)-1,3,5-triazine-2,4-diyl]bis[5-(2-ethylhexyl)oxy]phenol, marketed as Tinosorb S by BASF, N2,N4-bis[4-[5-(1,1-dimethylpropyl)-2-benzoxazolyl]phenyl]-N6-(2-ethylhexyl)-1,3,5-triazine-2,4,6-triamine marketed as Uvasorb K 2A by Sigma 3V, Bis(butylbenzoate)diaminotriazine aminopropyltrisiloxane (Mexoryl SBS).

Anthraniline derivatives: Menthyl anthranilate, for example marketed by Symrise under the name “Neo Heliopan MA”.

Imidazole derivatives: Ethylhexyldimethoxybenzylidenedioxoimidazoline propionate.

Benzalmalonate derivatives: polyorganosiloxanes containing functional benzalmalonate groups, such as, for example, polysilicone-15, for example marketed by Hoffmann LaRoche under the name “Parsol SLX”.

4,4-Diarylbutadiene derivatives: 1,1-Dicarboxy(2,2′-dimethylpropyl)-4,4-diphenylbutadiene.

Benzoxazole derivatives: 2,4-bis[5-(1-dimethylpropyl)benzoxazol-2-yl(4-phenyl)imino]-6-(2-ethylhexyl)imino-1,3,5-triazine, for example marketed by Sigma 3V under the name Uvasorb K2A, and mixtures comprising this.

Piperazine derivatives, such as, for example, the compound

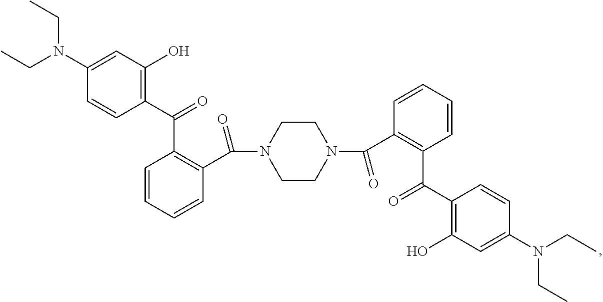 [SMILES: CCN(CC)c1ccc(C(=O)c2ccccc2C(=O)N2CCN(C(=O)c3ccccc3C(=O)c3ccc(N(CC)CC)cc3O)CC2)c(O)c1]

or the UV filters of the following structures

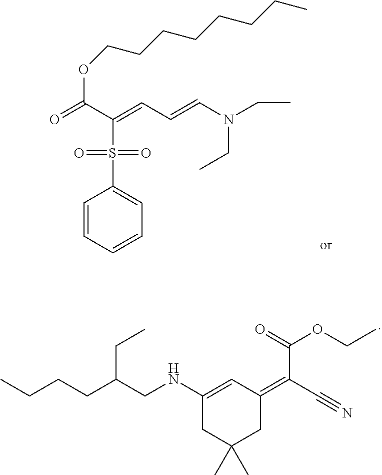

It is also possible to use UV filters based on polysiloxane copolymers having a random distribution in accordance with the following formula, where, for example, a=1,2; b=58 and c=2,8:

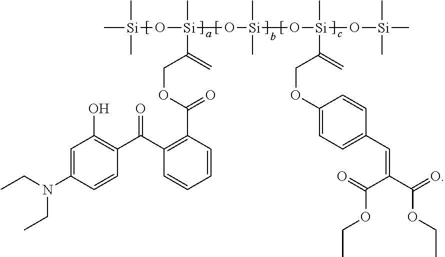

The compounds listed should only be regarded as examples. It is of course also possible to use other UV filters.

Suitable organic UV-protecting substances can preferably be selected from the following list: Ethylhexyl salicylate, Phenylbenzimidazolesulfonic acid, Benzophenone-3, Benzophenone-4, Benzophenone-5, n-Hexyl 2-(4-diethylamino-2-hydroxybenzoyl)benzoate, 4-Methylbenzylidenecamphor, Terephthalylidenedicamphorsulfonic acid, Disodium phenyldibenzimidazoletetrasulfonate, Methylenebis(benzotriazolyl)tetramethylbutylphenol, Ethylhexyl Triazone, Diethylhexyl Butamido Triazone, Drometrizole trisiloxane, Polysilicone-15, 1,1-Dicarboxy(2,2′-dimethylpropyl)-4,4-diphenylbutadiene, 2,4-bis[5-1 (dimethylpropyl)benzoxazol-2-yl(4-phenyl)imino]-6-(2-ethylhexyl)-imino-1,3,5-triazine and mixtures thereof.

These organic UV filters are generally incorporated into formulations in an amount of 0.01 percent by weight to 20 percent by weight, preferably 1% by weight −10% by weight.

Besides the extracts according to the invention and the optional organic UV lifters, as described above, the preparations may comprise further inorganic UV filters, so-called particulate UV filters.

These combinations with particulate UV filters are possible both as powder and also as dispersion or paste of the following types.

Preference is given here both to those from the group of the titanium dioxides, such as, for example, coated titanium dioxide (for example Eusolex® T-2000, Eusolex®T-AQUA, Eusolex®T-AVO, Eusolex®T-OLEO), zinc oxides (for example Sachtotec®), iron oxides or also cerium oxides and/or zirconium oxides.

Furthermore, combinations with pigmentary titanium dioxide or zinc oxide are also possible, where the particle size of these pigments are greater than or equal to 200 nm, for example Hombitan® FG or Hombitan® FF-Pharma.

It may furthermore be preferred for the preparations to comprise inorganic UV filters which have been aftertreated by conventional methods, as described, for example, in Cosmetics & Toiletries, February 1990, Vol. 105, pp. 53 64. One or more of the following aftertreatment components can be selected here: amino acids, beeswax, fatty acids, fatty acid alcohols, anionic surfactants, lecithin, phospholipids, sodium, potassium, zinc, iron or aluminium salts of fatty acids, polyethylenes, silicones, proteins (particularly collagen or elastin), alkanolamines, silicon dioxide, aluminium oxide, further metal oxides, phosphates, such as sodium hexametaphosphate, or glycerine.

Particulate UV filters which are preferably employed here are:
untreated titanium dioxides, such as, for example, the products Microtitanium Dioxide MT 500 B from Tayca; titanium dioxide P25 from Degussa,
Aftertreated micronised titanium dioxides with aluminium oxide and silicon dioxide aftertreatment, such as, for example, the product “Microtitanium Dioxide MT 100 SA from Tayca; or the product “Tioveil Fin” from Uniqema,
Aftertreated micronised titanium dioxides with aluminium oxide and/or aluminium stearate/laurate aftertreatment, such as, for example, Microtitanium Dioxide MT 100 T from Tayca, Eusolex T-2000 from Merck,
Aftertreated micronised titanium dioxides with iron oxide and/or iron stearate aftertreatment, such as, for example, the product “Microtitanium Dioxide MT 100 F” from Tayca,
Aftertreated micronised titanium dioxides with silicon dioxide, aluminium oxide and silicone aftertreatment, such as, for example, the product “Microtitanium Dioxide MT 100 SAS”, from Tayca,
Aftertreated micronised titanium dioxides with sodium hexametaphosphates, such as, for example, the product “Microtitanium Dioxide MT 150 W” from Tayca.

The treated micronised titanium dioxides employed for the combination may also be aftertreated with:
octyltrimethoxysilanes; such as, for example, the product Tego Sun T 805 from Degussa,
silicon dioxide; such as, for example, the product Parsol T-X from DSM,
aluminium oxide and stearic acid; such as, for example, the product UV-Titan M160 from Sachtleben,
aluminium and glycerine; such as, for example, the product UV-Titan from Sachtleben,
aluminium and silicone oils, such as, for example, the product UV-Titan M262 from Sachtleben,
sodium hexametaphosphate and polyvinylpyrrolidone,
polydimethylsiloxanes, such as, for example, the product 70250 Cardre UF TiO2SI3″ from Cardre,
polydimethylhydrogenosiloxanes, such as, for example, the product Microtitanium Dioxide USP Grade Hydrophobic” from Color Techniques.

The combination with the following products may furthermore also be advantageous:
Untreated zinc oxides, such as, for example, the product Z-Cote from BASF (Sunsmart), Nanox from Elementis
aftertreated zinc oxides, such as, for example, the following products:
   “Zinc Oxide CS-5” from Toshibi (ZnO aftertreated with polymethylhydrogenosiloxanes)
   Nanogard Zinc Oxide FN from Nanophase Technologies
   “SPD-Z1” from Shin-Etsu (ZnO aftertreated with a silicone-grafted acrylic polymer, dispersed in cyclodimethylsiloxanes
   “Escalol Z100” from ISP (aluminium oxide-aftertreated ZnO dispersed in an ethylhexyl methoxycinnamate/PVP-hexadecene/methicone copolymer mixture)
   “Fuji ZNO-SMS-10” from Fuji Pigment (ZnO aftertreated with silicon dioxide and polymethylsilesquioxane);
   Untreated cerium oxide micropigment, for example with the name “Colloidal Cerium Oxide” from Rhone Poulenc
   Untreated and/or aftertreated iron oxides with the name Nanogar from Arnaud.

By way of example, it is also possible to employ mixtures of various metal oxides, such as, for example, titanium dioxide and cerium oxide, with and without aftertreatment, such as, for example, the product Sunveil A from Ikeda. In addition, it is also possible to use mixtures of aluminium oxide, silicon dioxide and silicone-aftertreated titanium dioxide. zinc oxide mixtures, such as, for example, the product UV-Titan M261 from Sachtleben.

These inorganic UV filters are generally incorporated into the preparations in an amount of 0.1 percent by weight to 25 percent by weight, preferably 2% by weight to 10% by weight.

By combination of one or more of the said compounds having a UV filter action, the protective action against harmful effects of the UV radiation can be optimised.

All said UV filters can also be employed in encapsulated form. In particular, it is advantageous to employ organic UV filters in encapsulated form.

The capsules in preparations to be employed in accordance with the invention are preferably present in amounts which ensure that the encapsulated UV filters are present in the preparation in the percent by weight ratios indicated above.

The preparations described which comprise the extract according to the invention may furthermore also comprise coloured pigments, where the layer structure of the pigments is not limited.

The coloured pigment should preferably be skin-coloured or brownish on use of 0.5 to 5% by weight. The selection of a corresponding pigment is familiar to the person skilled in the art.

Preferred preparations may likewise comprise at least one further cosmetic active compound, for example selected from antioxidants, anti-ageing, antiwrinkle, anti-dandruff, anti-acne, anti-cellulite active compounds, deodorants or vitamins.

The protective action of preparations against oxidative stress or against the effect of free radicals can be improved if the preparations comprise one or more antioxidants, the person skilled in the art being presented with absolutely no difficulties in selecting antioxidants which act suitably quickly or with a time delay.

There are many proven substances known from the specialist literature which can be used as antioxidants, for example amino acids (for example glycine, histidine, tyrosine, tryptophan) and derivatives thereof, imidazoles, (for ecample urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents, (for example α-hydroxyfatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA, pentasodium ethylenediamine tetramethylene phosphonate and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Suitable antioxidants are also compounds of the formulae A or B

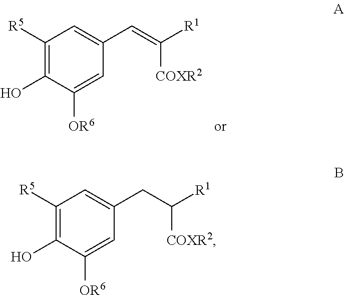

in which
R¹ can be selected from the group —C(O)CH₃, —CO₂R³, —C(O)NH₂ and —C(O)N(R⁴)₂,
X denotes O or NH,
R² denotes linear or branched alkyl having 1 to 30 C atoms,
R³ denotes linear or branched alkyl having 1 to 20 C atoms,
R⁴ in each case, independently of one another, denotes H or linear or branched alkyl having 1 to 8 C atoms,
R⁵ denotes H, linear or branched alkyl having 1 to 8 C atoms or linear or branched alkoxy having 1 to 8 C atoms and
R⁶ denotes linear or branched alkyl having 1 to 8 C atoms, preferably derivatives of 2-(4-hydroxy-3,5-di methoxybenzylidene)malonic acid and/or 2-(4-hydroxy-3,5-di methoxybenzyl)malonic acid, particularly preferably bis(2-ethylhexyl) 2-(4-hydroxy-3,5-dimethoxybenzylidene)malonate (for example Oxynex® ST Liquid) and/or bis(2-ethylhexyl) 2-(4-hydroxy-3,5-dimethoxybenzyl)malonate (for example RonaCare® AP).

Mixtures of antioxidants are likewise suitable for use in the preparations according to the invention. Known and commercial mixtures are, for example, mixtures comprising, as active ingredients, lecithin, L-(+)-ascorbyl palmitate and citric acid, natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed in the reparations according to the invention in amounts of 0.1 to 20% by weight, preferably in amounts of 0.1 to 10% by weight.

Of the phenols which can be used in accordance with the invention, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydoxyflavones. It is observed therein that dihydroxyflavones containing an OH group adjacent to the keto function or OH groups in the 3′4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers and I. M. C. M. Rietjens (Free Radical Biology&Medicine 2001, 31(7), 869-881, have investigated the pH dependence of the antioxidant action of hydroxyflavones. Quercetin exhibits the highest activity amongst the structures investigated over the entire pH range.

Suitable anti-ageing active compounds, in particular for skin-care preparations, are preferably so-called compatible solutes. These are substances which are involved in the osmosis regulation of plants or microorganisms and can be isolated from these organisms. The generic term compatible solutes here also encompasses the osmolytes described in German patent application DE-A-10133202. Suitable osmolytes are, for example, the polyols, methylamine compounds and amino acids and respective precursors thereof. Osmolytes in the sense of German patent application DE-A-10133202 are taken to mean, in particular, substances from the group of the polyols, such as, for example, myo-inositol, mannitol or sorbitol, and/or one or more of the osmolytically active substances mentioned below: taurine, choline, betaine, phosphorylcholine, glycerophosphorylcholines, glutamine, glycine, α-alanine, glutamate, aspartate, proline and taurine. Precursors of these substances are, for example, glucose, glucose polymers, phosphatidylcholine, phosphatidylinositol, inorganic phosphates, proteins, peptides and polyamino acids. Precursors are, for example, compounds which are converted into osmolytes by metabolic steps.

Compatible solutes which are preferably employed in accordance with the invention are substances selected from the group consisting of pyrimidinecarboxylic acids (such as ectoin and hydroxyectoin), proline, betaine, glutamine, cyclic diphosphoglycerate, N.-acetylornithine, trimethylamine Noxide di-myo-inositol phosphate (DIP), cyclic 2,3-diphosphoglycerate (cDPG), 1,1-diglycerol phosphate (DGP), β-mannosyl glycerate (firoin), β-mannosyl glyceramide (firoin-A) or/and dimannosyl diinositol phosphate (DMIP) or an optical isomer, derivative, for example an acid, a salt or ester, of these compounds, or combinations thereof.

Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid) and derivatives thereof.

Additionally, anti-aging active compounds which can be used are products from Merck, such as, for example, 5,7-dihydroxy-2-methylchromone, marketed under the trade name RonaCare® Luremine, RonaCare® Isoquercetin, RonaCare® Tilirosid or RonaCare® Cyclopeptide 5.

The preparations to be employed may comprise vitamins as further ingredients. Preference is given to vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active compound), thiamine (vitamin B₁), nicotinic acid (niacin), pyridoxine, pyridoxal, pyridoxamine, (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. In the case of cosmetic application, vitamins are usually added with the flavonoid-containing premixes or preparations in ranges from 0.01 to 5.0% by weight, based on the total weight. Nutrition-physiological applications are oriented towards the respective recommended vitamin requirement.

The retinoids described are at the same time also effective anti-cellulite active compounds. A likewise known anti-cellulite active compound is caffeine.

The present invention also relates to a process for the preparation of a preparation, as described above, characterised in that the extract from plant parts of Darlingtonia californica is mixed with a vehicle which is suitable for topical preparations. The mixing can optionally also take place with further assistants and or fillers. Suitable vehicles and assistants or fillers are described in detail in the following part.

The said constituents of the preparation can be incorporated in the usual manner, with the aid of techniques which are well known to the person skilled in the art.

The cosmetic and dermatological preparations can be in various forms. Thus, they can be, for example, a solution, a water-free preparation, an emulsion or microemulsion of the water-in-oil (W/O) type or of the oil-in-water (O/W) type, a multiple emulsion, for example of the water-in-oil-in-water (W/O/W) or O/W/O type, a gel, a solid stick, an ointment or also an aerosol. Preference is given to emulsions. O/W emulsions are particularly preferred. Emulsions, W/O emulsions and O/W emulsions can be obtained in the usual manner.

The following, for example, may be mentioned as application form of the preparations to be employed: solutions, suspensions, emulsions, PIT emulsions, pastes, ointments, gels, creams, lotions, powders, soaps, surfactant-containing cleansing preparations, oils, aerosols plasters, compresses, bandages and sprays.

Preferred assistants originate from the group of preservatives, stabilisers, solubilisers, colorants, odour improvers.

Ointments, pastes, creams and gels may comprise the customary vehicles which are suitable for topical application, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary vehicles, for example lactose, talc, silica, aluminium hydroxide, calcium silicate and polyamide powder, or mixtures of these substances. Sprays may additionally comprise the customary readily volatile, liquefied propellants, for example chlorofluorocarbons, propane/butane or dimethyl ether. Compressed air can also advantageously be used.

Solutions and emulsions may comprise the customary vehicles, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, XTend 226 (L'Oréal), glycerol fatty acid esters, polyethylene glycols and fatty acid esters of sorbitan, or mixtures of these substances.

A preferred solubiliser in general is 2-isopropyl-5-methylcyclohexanecarbonyl-D-alanine methyl ester.

Suspensions may comprise the customary vehicles, such as liquid diluents, for example water, ethanol or propylene glycol, suspension media, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary vehicles, such as alkali metal salts of fatty acids, salts of fatty acid monoesters, fatty acid protein hydrolysates, isothionates, lanolin, fatty alcohol, vegetable oils, plant extracts, glycerol, sugars, or mixtures of these substances.

Surfactant-containing cleansing products may comprise the customary vehicles, such as salts of fatty alcohol sulfates, fatty alcohol ether sulfates, sulfosuccinic acid monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances.

Face and body oils may comprise the customary vehicles, such as synthetic oils, such as fatty acid esters, fatty alcohols, silicone oils, natural oils, such as vegetable oils and oily plant extracts, paraffin oils, lanolin oils, or mixtures of these substances.

Further typical cosmetic application forms are also lipsticks, lip-care sticks, powder make-up, emulsion make-up and wax make-up, and sunscreen, pre-sun and after-sun preparations.

The preferred preparation forms also include, in particular, emulsions.

Emulsions are advantageous and comprise, for example, the said fats, oils, waxes and other fatty substances, as well as water and an emulsifier, as usually used for a preparation of this type.

The lipid phase may advantageously be selected from the following group of substances:
mineral oils, mineral waxes
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, or from the group of esters of aromatic carboxylic acid and saturated and/or unsaturated, branched and/or unbranched alcohols having a chain length of 3 to 30 C atoms. Ester oils of this type can then advantageously be selected from the group isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group branched and unbranched hydrocarbons and hydrocarbon waxes, silicone oils, dialkyl ethers, the group of saturated or unsaturated, branched or unbranched alcohols, and fatty acid triglycerides, specifically the triglycerol esters of saturated and/or unsaturated, branched and/or unbranched alkanecarboxylic acids having a chain length of 8 to 24, in particular 12-18 C atoms. The fatty acid triglycerides may, for example, advantageously be selected from the group of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as sole lipid component of the oil phase.

The aqueous phase of the preparations to be employed optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol, glycerol, and, in particular, one or more thickeners, which may advantageously be selected from the group silicon dioxide, aluminium silicates, polysaccharides and derivatives thereof, for example hyaluronic acid, xanthan gum, hydroxypropylmethylcellulose, particularly advantageously from the group of the polyacrylates, preferably a polyacrylate from the group of the so-called Carbopols, for example Carbopol grades 980, 981, 1382, 2984, 5984, in each case individually or in combination.

In particular, mixtures of the above-mentioned solvents are used. In the case of alcoholic solvents, water may be a further constituent.

In a preferred embodiment, the preparations to be employed comprise hydrophilic surfactants. The hydrophilic surfactants are preferably selected from the group of the alkylglucosides, acyl lactylates, betaines and coconut amphoacetates.

Emulsifiers that can be used are, for example, the known W/O and O/W emulsifiers. It is advantageous to use further conventional co-emulsifiers in the preferred O/W emulsions.

The co-emulsifiers selected are advantageously, for example, O/W emulsifiers, principally from the group of substances having HLB values of 11-16, very particularly advantageously having HLB values of 14.5-15.5, so long as the O/W emulsifiers have saturated radicals R and R′. If the O/W emulsifiers have unsaturated radicals R and/or R′, or if isoalkyl derivatives are present, the preferred HLB value of such emulsifiers may also be lower or higher.

It is advantageous to select the fatty alcohol ethoxylates from the group of the ethoxylated stearyl alchols, cetyl alcohols, cetylstearyl alcohols (cetearyl alcohols).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostearate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

An ethoxylated alkyl ether carboxylic acid or salt thereof which can advantageously be used is sodium laureth-11 carboxylate. An alkyl ether sulfate which can advantageously be used is sodium laurethl-4 sulfate. An ethoxylated cholesterol derivative which can advantageously be used is polyethylene glycol (30) cholesteryl ether. Polyethylene glycol (25) soyasterol has also proven successful. Ethoxylated triglycerides which can advantageously be used are the polyethylene glycol (60) evening primrose glycerides.

It is furthermore advantageous to select the polyethylene glycol glycerol fatty acid esters from the group polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/cprinate, polyethylene glycol (20) glyceryl oleate, polyethylene glycol (20) glyceryl isostearate, polyethylene glycol (18) glyceryl oleate (cocoate).

It is likewise favourable to select the sorbitan esters from the group polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

The following can be employed as optional W/O emulsifiers, but ones which may nevertheless be advantageous in accordance with the invention: fatty alcohols having 8 to 30 carbon atoms, monoglycerol esters of saturated and/or unsaturated, branched and/or unbranched alkanecarboxylic acids having a chain length of 8 to 24, in particular 12-18 C atoms, diglycerol esters of saturated and/or unsaturated, branched and/or unbranched alkanecarboxylic acids having a chain length of 8 to 24, in particular 12-18 C atoms, monoglycerol ethers of saturated and/or unsaturated, branched and/or unbranched alcohols having a chain length of 8 to 24, in particular 12-18 C atoms, diglycerol ethers of saturated and/or unsaturated, branched and/or unbranched alcohols having a chain length of 8 to 24, in particular 12-18 C atoms, propylene glycol esters of saturated and/or unsaturated, branched and/or unbranched alkanecarboxylic acids having a chain length of 8 to 24, in particular 12-18 C atoms, and sorbitan esters of saturated and/or unsaturated, branched and/or unbranched alkanecarboxylic acids having a chain length of 8 to 24, in particular 12-18 C atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate, glyceryl monocaprylate or PEG-30 dipolyhydroxystearate.

The preparation may comprise cosmetic adjuvants which are usually used in this type of preparation, such as, for example, thickeners, softeners, moisturisers, surface-active agents, emulsifiers, preservatives, antifoams, perfumes, waxes, lanolin, propellants, dyes and/or pigments, and other ingredients usually used in cosmetics.

The dispersant or solubiliser used can be an oil, wax or other fatty bodies, a lower monoalcohol or a lower polyol or mixtures thereof. Particularly preferred monoalcohols or polyols include ethanol, i-propanol, propylene glycol, glycerol and sorbitol.

A preferred embodiment of the invention is an emulsion which is in the form of a protective cream or milk and comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The preparation may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a preparation is formulated as an aerosol, use is generally made of the customary propellants, such as alkanes, fluoroalkanes and chlorofluoroalkanes, preferably alkanes.

Even without further comments, it is assumed that a person skilled in the art will be able to utilise the above description in the broadest scope. The preferred embodiments and examples should therefore merely be regarded as descriptive disclosure which is absolutely not limiting in any way. The complete disclosure content of all applications and publications mentioned above and below is incorporated into this application by way of reference.

The examples are intended to explain the present invention in greater detail without restricting the scope thereof.

### EXAMPLES

### Example 1

### Ethanol Extract from Darlingtonia californica

2.8 g of tubular leaves of Darlingtonia californica are dried to constant weight at 40° C. and 200 mbar in a vacuum drying cabinet, comminuted roughly and extracted with 250 ml of ethanol under reflux and filtered off. After removal of the solvent, 497 mg of extract are obtained.

### Example 2

### Performance of the B16 V Mouse Melanoma Cell Test

B16V mouse melanoma cells (manufacturer: DSMZ; Article No.: ACC370) are transferred into RPMI medium (Invitrogen, Article No.: 31870), to which 10% of FBS (foetal bovine serum; Invitrogen, Article No.: 10499044), 2 mM L-glutamine (Invitrogen, Article No: 25030) and 1 mM sodium pyruvate (Invitrogen, Article No.: 11360) had additionally been added, and incubated at 37° C. and 5% CO₂ for 72 h. The medium is separated off, and the cells are washed once with 10 ml of DPBS (Dulbecco's phosphate-buffered salines; Invitrogen, Article No.: 14190), and the medium is subsequently removed by suction. 1 ml of HyQtase cell detachment solution (Hyclone, Article No.: SV30030.01) is added to the cells. The bottle is swirled a number of times, and the HyQtase cell detachment solution is subsequently removed by suction. The cells are then incubated in the incubator at 37° C. and 5% CO₂ for 5 min. The cells are taken up in the modified RPMI medium (see above), and the cell count is determined. To this end, the cells are stained with Trypan Blue and counted in a Neubauer counting chamber. The cells are subsequently sown out again in the modified RPMI medium (see above) in a defined cell count of 80,000 cells per well (6-well clear plate, TCT, PS (Nunc)).

The cells are incubated at 37° C. and 5% CO₂ for 24 h, the medium is then removed. 1980 μl of the substance dilution are subsequently added. For this substance dilution, the extract from Example 1 is dissolved in DMSO and subsequently filtered through a sterile filter (0.2 μm, Millipore, Article No. SLLG013SL). The solution is then diluted with the modified RPMI medium (see above, but in this case the FBS content is only 5%) in such a way that the final concentration of the extract is 0.1 mg/ml or 9.95 mg/ml.

20 μl of an alpha-MSH solution (alpha-melanocyte-stimulating hormone, DMSO, Sigma, Article No.: D2650) are then added, so that the alpha-MSH concentration in the well is 10⁻⁸ M. The plate is subsequently incubated again at 37° C. and 5% CO₂ for 24 h. The process described in this section is repeated a further twice in total.

After the final incubation period, the medium is removed by suction, and the cells are washed with 1000 μl of DPBS (Invitrogen, Article No.: 14190). The medium is again removed by suction. 250 μl of HyQtase cell detachment solution (Hyclone, Article No.: SV30030.01) are added to the cells. The 6-well plate is swirled a number of times, and the HyQtase cell detachment solution is subsequently removed by suction. The cells are then incubated in the incubator at 37° C. and 5% CO₂ for 5 min. The cells are taken up in 1.5 ml of DPBS (Invitrogen, Article No.: 14190) and transferred into a cup (SARSTEDT, Ref. 72.692.005). The cell count is subsequently determined. To this end, the cells are stained with Trypan Blue and counted in a Neubauer counting chamber. The cells centrifuged for 1 min at 3500 g. The pellets obtained are photographed, and the supernatant is subsequently removed by suction. The pellets are dissolved in 1 ml of 1N NaOH at 80° C. for 1 h and then cooled to RT. Four times 200 μl per cup (as quadruple determination) are subsequently pipetted into a 96-well plate (VWR, Article No.: 4100636981), and the absorption at a wavelength of 405 nm is determined (Safire, Tecan). The content of melanin can be determined in this way by means of a calibration line.

As comparison, a sample without extract, but with 0.1% of DMSO or with 0.1% of DMSO and alpha-MSH (concentration in the well at 10⁻⁸ M) is used in parallel in each case.

Results:

The extract from Darlingtonia californica from Example 1 causes an increase in the melanin content both in the unstimulated melanocytes and also in the melanocytes stimulated to tanning by α-MSH. This means that the extracts according to the invention are even capable of stimulating cells which have already been stimulated by α-MSH additionally to synthesise melanin further.

TABLE 1
Content of melanin per cell:

| | Melanin content [pg/cell] |
|---|---|
| DMSO (0.1%) | 13.4 |
| DMSO (0.1%) + α-MSH | 35.7 |
| 100 μg extract from Example 1 + α-MSH | 51.8 |
| 100 μg extract from Example 1 | 20.1 |

### Example 3

### O/W Formulation

| | Source | | |
|---|---|---|---|
| Constituents/ | of | | [% by |
| trade name | supply | INCI | wt.] |
| A | | | |
| Marlipal 1618/11 | (1) | CETEARETH-11 | 3 |
| Lanette O | (2) | CETEARYL- | 7 |
| | | ALCOHOL | |
| Luvitol EHO | (3) | CETEARYL- | 5 |
| | | OCTANOATE | |
| Tegosoft TN | (4) | C12-15 | 2.5 |
| | | ALKYLBENZOATE | |
| Miglyol 812 N | (1) | CAPRYLIC/CAPRIC | 2.5 |
| | | TRIGLYCERIDE | |
| Propyl 4- | (5) | PROPYLPARABEN | 0.05 |
| hydroxybenzoate | | | |
| Extract from Example 1 | | | 0.5 |
| B | | | |
| 1,2-Propanediol | (5) | PROPYLENE | 4 |
| | | GLYCOL | |
| Methyl 4- | (5) | METHYLPARABEN | 0.15 |
| hydroxybenzoate | | | |
| Water, demineralised | | AQUA (WATER) | to 100 |

| | |
|---|---|
| Total | 100.00 |

Preparation Process:

Firstly, phase A is warmed to 75° C. and phase B to 80° C. Phase B is then slowly added to phase A with stirring and stirred until a homogeneous mixture forms.

Sources of Supply:

(1) Sasol Germany GmbH (2) Cognis GmbH (3) BASF AG (4) Degussa-Goldschmidt AG (5) Merck KGaA/Rona®

### Example 4

### O/W Formulation

| | Source | | |
|---|---|---|---|
| Constituents/ | of | | [% by |
| trade name | supply | INCI | wt.] |
| A | | | |
| Marlipal 1618/11 | (1) | CETEARETH-11 | 3 |
| Lanette O | (2) | CETEARYL- | 7 |
| | | ALCOHOL | |
| Luvitol EHO | (3) | CETEARYL- | 5 |
| | | OCTANOATE | |
| Tegosoft TN | (4) | C12-15 | 2.5 |
| | | ALKYLBENZOATE | |
| Miglyol 812 N | (1) | CAPRYLIC/CAPRIC | 2.5 |
| | | TRIGLYCERIDE | |
| Propyl 4- | (5) | PROPYLPARABEN | 0.05 |
| hydroxybenzoate | | | |
| B | | | |
| 1,2-Propanediol | (5) | PROPYLENE | 4 |
| | | GLYCOL | |
| Methyl 4- | (5) | METHYLPARABEN | 0.15 |
| hydroxybenzoate | | | |
| Extract from Example 1 | | | 1.0 |
| Water, demineralised | | AQUA (WATER) | to 100 |

| | |
|---|---|
| Total | 100.00 |

Preparation Process:

Firstly, phase A is warmed to 75° C. and phase B to 80° C. Phase B is then slowly added to phase A with stirring and stirred until a homogeneous mixture forms.

Sources of Supply:

(1) Sasol Germany GmbH (2) Cognis GmbH (3) BASF AG (4) Degussa-Goldschmidt AG (5) Merck KGaA/Rona®

### Example 5

### O/W Formulation

| | Source | | |
|---|---|---|---|
| Constituents/ | of | | [% by |
| trade name | supply | INCI | wt] |
| A | | | |
| Tego Care 150 | (1) | GLYCERYL STEARATE, | 8 |
| | | STEARETH-25, | |
| | | CETETH-20, | |
| | | STEARYL ALCOHOL | |
| Lanette O | (2) | CETEARYL ALCOHOL | 1.5 |
| Luvitol EHO | (3) | CETEARYL | 5 |
| | | OCTANOATE | |
| Miglyol 812 N | (4) | CAPRYLIC/CAPRIC | 5 |
| | | TRIGLYCERIDE | |
| Paraffin liquid | (5) | PARAFFINUM | 3 |
| | | LIQUIDUM | |
| | | (MINERAL OIL) | |
| AbilWax 2434 | (1) | STEAROXY | 1.6 |
| | | DIMETHICONE | |
| Dow Corning 200 Fluid | (6) | DIMETHICONE | 0.5 |
| (350 cs) | | | |
| Propyl 4- | (5) | PROPYLLPARABEN | 0.05 |
| hydroxybenzoate | | | |
| B | | | |
| 1,2-Propanediol | (5) | PROPYLENE GLYCOL | 3 |
| Methyl 4- | (5) | METHYLPARABEN | 0.15 |
| hydroxybenzoate | | | |
| Water, demineralised | | AQUA (WATER) | to 100 |
| C | | | |
| Probiol L 05018 (empty | (7) | AQUA, ALCOHOL | 5 |
| liposomes) | | DENAT, LECITHIN, | |
| | | GLYCERINE, DISODIUM | |
| | | PHOSPHATE | |
| Water, demineralised | | AQUA (WATER) | 10.00 |
| Extract from Example 1 | | | 1.0 |

| | |
|---|---|
| Total | 100.00 |

Preparation Process:

Firstly, phases A and B are warmed to 80° C. Phase B is then slowly added to phase A with stirring and homogenised. The mixture is then cooled, and phase C is added at 40° C.

Sources of Supply:

(1) Degussa-Goldschmidt AG, (2) Cognis GmbH, (3) BASF AG, (4) Sasol Germany GmbH, (5) Merck KGaA/Rona®, (6) Dow Corning, (7) Kuhs GmbH & Co. KG

### Example 6

### W/O Formulation

| | Source | | |
|---|---|---|---|
| Constituents/ | of | | [% by |
| trade name | supply | INCI | wt.] |
| A | | | |
| Dow Corning 3225 C | (1) | CYCLOMETHICONE, | 23.6 |
| | | DIMETHICONE | |
| | | COPOLYOL | |
| Propyl 4- | (2) | PROPYLPARABEN | 0.05 |
| hydroxybenzoate | | | |
| B | | | |
| Extract from Example 1 | | | 2.0 |
| Methyl 4- | (2) | METHYLPARABEN | 0.15 |
| hydroxybenzoate | | | |
| 1,2-Propanediol | (2) | PROPYLENE GLYCOL | 35.9 |
| Water, demineralised | | AQUA (WATER) | to 100 |

| | |
|---|---|
| Total | 100.00 |

Preparation Process:

Firstly, phase B is dissolved and then added to phase A. The pH is adjusted to the value pH=6.0 using sodium hydroxide solution or citric acid.

Sources of Supply:

(1) Dow Corning (2) Merck KGaA/Rona®

### Example 7

### O/W Anti-Ageing Cream with UV a/B Protection

| | Source | | |
|---|---|---|---|
| Constituents/ | of | | [% by |
| trade name | supply | INCI | wt.] |
| A | | | |
| Eusolex ® 2292 | (1) | ETHYLHEXYL | 3 |
| | | METHOXYCINNAMATE, | |
| | | BHT | |
| Eusolex ® 4360 | (1) | BENZOPHENONE-3 | 0.5 |
| Tego Care 150 | (2) | GLYCERYL STEARATE, | 8 |
| | | STEARETH-25, CETETH- | |
| | | 20, STEARYL ALCOHOL | |
| Lanette O | (3) | CETEARYL ALCOHOL | 1.5 |
| Luvitol EHO | (4) | CETEARYL | 5 |
| | | OCTANOATE | |
| Miglyol 812 N | (5) | CAPRYLIC/CAPRIC | 5 |
| | | TRIGLYCERIDE | |
| Paraffin liquid | (1) | PARAFFINUM | 3 |
| | | LIQUIDUM | |
| | | (MINERAL OIL) | |
| Abil-Wax 2434 | (2) | STEAROXY | 1.6 |
| | | DIMETHICONE | |
| Dow Corning 200 Fluid | (6) | DIMETHICONE | 0.5 |
| (350 cs) | | | |
| Propyl 4- | (1) | PROPYLPARABEN | 0.05 |
| hydroxybenzoate | | | |
| B | | | |
| 1,2-Propanediol | (1) | PROPYLENE GLYCOL | 3 |
| Methyl 4- | (1) | SODIUM | 0.17 |
| hydroxybenzoate | | METHYLPARABEN | |
| sodium salt | | | |
| Extract from Example 1 | | | 0.5 |
| Water, demineralised | | AQUA (WATER) | to 100 |

| | |
|---|---|
| Total | 100.00 |

Preparation Process:

Firstly, phases A and B are mixed separately and warmed to 80° C. Phase B is then slowly added to phase A with stirring. The mixture is homogenised cooled to room temperature.

Sources of Supply:

(1) Merck KGaA/Rona®, (2) Degussa-Goldschmidt AG, (3) Cognis GmbH, (4) BASF AG, (5) Sasol Germany GmbH, (6)

1.-8. (canceled)

9. A method of self-tanning the skin of a human subject, comprising applying an extract from plant parts of Darlingtonia californica to the skin of the human subject.

10. A method for increasing melanin synthesis, improving melanin transport and/or improving the distribution of melanin in suprabasal layers of human skin, comprising applying an extract an extract from plant parts of Darlingtonia californica to the skin.

11. The method of claim 9, wherein the plant parts are leaves.

12. The method of claim 16, wherein the preparation comprises 0.01 to 99% by weight of the extract, based on the total weight of the preparation.

13. The method of claim 16, wherein at least one further self-tanning substance is present.

14. (canceled)

15. The method of claim 10, wherein the plant parts are leaves.

16. The method of claim 9, wherein the extract is in the form of a cosmetic or dermatological preparation.

17. The method of claim 10, wherein the extract is in the form of a cosmetic or dermatological preparation.

18. The method of claim 17, wherein the preparation comprises 0.01 to 99% by weight of the extract, based on the total weight of the preparation.

19. The method of claim 17, wherein at least one further self-tanning substance is present.

20. The method of claim 16, wherein the preparation comprises further assistants and/or fillers.

21. The method of claim 17, wherein the preparation comprises further assistants and/or fillers.
